(19) United States
(12) Patent Application Publication
(10) Pub. No.: US 2021/0299362 A1
(43) Pub. Date: Sep. 30, 2021

(54) NEEDLE ASSEMBLY HAVING A STERILE INTERIOR

(71) Applicant: Novo Nordisk A/S, Bagsvaerd (DK)

(72) Inventors: Abbas Atraktchi, Frederiksberg (DK); Andre Larsen, Dragoer (DK)

(21) Appl. No.: 17/260,500

(22) PCT Filed: Jul. 5, 2019
(86) PCT No.: PCT/EP2019/068081

§ 371 Date: Jan. 14, 2021

(30) Foreign Application Priority Data
Jul. 18, 2018 (EP) 18184134.7

(51) Int. Cl. A61M 5/32

(52) U.S. Cl. CPC A61M 5/3202; A61M 2205/02; A61M 2207/00

(57) ABSTRACT

The invention relates to a needle assembly, comprising a needle hub with a sterility barrier with a matrix of tear lines to enhance puncture of the sterility barrier. The matrix of tear lines consists of a plurality of individual tear lines defining a plurality of flaps which each are bendable into the interior of the needle hub when the sterility barrier is punctured.

### THE TECHNICAL FIELD OF THE INVENTION

The invention relates to a needle assembly and especially such needle assembly wherein a needle hub houses a proximal part of a needle cannula in a sterile interior which is covered by a breakable sterility barrier.

### DESCRIPTION OF RELATED ART

When attaching a needle assembly to an injection device the user usually has to manually remove a sterility barrier before the needle assembly is attached to the injection device. However, in some injection devices there is no access to the needle hub and to the sterility barrier.

An example of such injection device is provided in FIGS. 14 and 15 of WO 2014/064100. In such injection device, the user attaches the needle assembly by moving some outer parts of the injection device. During this movement the proximal end of the needle cannula penetrates through a sterility barrier.

Another example of attaching a needle assembly to an injection device without prior removal of the sterility barrier is provided in EP 2,420,278. The needle hub depicted e.g. on the FIGS. 15 and 16 and on the FIGS. 38 and 39 has an open end which is sealed by a sterility barrier. This sterility barrier is embossed with a tear line which forms a circular portion such that the circular portion is punched out and moved along a longitudinal axis of the needle cannula when the sterility barrier is punctured by the injection delivery device.

Since the proximal tip of the needle cannula penetrates through the sterility barrier, fragments of the sterility barrier can enter into the lumen of the needle cannula and henceforth obstruct the free passage through the lumen when an injection later is being performed.

### DESCRIPTION OF THE INVENTION

It is henceforth an object of the present invention to provide a needle assembly with a breakable sterility barrier in which it is avoided that fragments enter into the lumen of the needle cannula during puncture of the sterility barrier.

Accordingly, in one aspect of the present invention, a needle assembly is provided which comprises a needle hub which has a surrounding wall with a distal part and a proximal part. This surrounding wall is preferably cylindrical and surrounds at least the proximal part of a needle cannula and is distally closed by a radially closed wall partition.

This radially closed wall partition further secures a needle cannula and forms, together with the surrounding wall, a cup-shaped interior.

The needle cannula is secured to the radially closed wall partition and has a distal end which extend distally from the radially closed wall partition and a proximal end which extend proximally from the radially closed wall partition. The distal end preferably penetrates through the skin of a user during injection and the proximal end penetrates into a cartridge such that a liquid drug present in an injection device is able to flow though the lumen of the needle cannula and into the user during injection.

The opening formed at the proximal part of the surrounding wall is sealed by a sterility barrier such that the surrounding wall, the radially closed wall partition securing the needle cannula and the sterility barrier together defined an interior which is sterile and houses the proximal end of the needle cannula.

The sterility barrier is further provided with a matrix of tear lines to enhance puncture of the sterility barrier and the matrix of tear lines consist of a plurality of individual tear lines which together defines a plurality of flaps which flaps are bendable into the interior of the needle hub upon puncture of the sterility barrier.

The flaps preferably has a triangular shape with one angle of the triangle being position in the centre (X) of the needle hub such that the individual flaps are able to bend inwardly into the interior of the needle hub without obtaining contact with the most proximal end of the needle cannula. Also, the length of each of the individual tear lines in the matrix is such that each flap stays out of contact with the proximal end of the needle cannula when the flaps are bended.

Henceforth, as the needle cannula does not contact the sterility barrier no fragments is being sliced off to enter into the lumen of the needle cannula. In example three or four such flaps could be provided however any number is possible.

The tear lines are preferably carved or embossed into the sterility barrier which barrier is made from a piece of paper, from a metallic foil which can be coated e.g. with a polymer or from a suitable polymeric foil.

The sterility barrier is connected to the proximal part of the needle hub and defines a thickness. In one example, the sterility barrier is made from two individual layers which thus make up the full thickness. In such example, the tear lines only extend through e.g. one of the layers. In any case, the depth of the tear lines are such that sterility of the interior of the needle hub can be maintained i.e. the tear lines do not extend through the full thickness of the sterility barrier.

In one example the individual tear lines can have a depth that differentiates such that the pattern in which the different tear lines breaks can be predetermined when designing the tear lines.

The needle assembly is further mounted on an injection device which comprises a cartridge. The coupling between the needle assembly and the injection device and/or the cartridge is preferably done by penetrating the injection device and/or the cartridge through the sterility barrier of the needle assembly such that the proximal end of the needle cannula connects to the cartridge without the proximal end of the needle cannula contacting the sterility barrier.

### DEFINITIONS

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel but could also be made from a polymeric material or a glass material. The needle cannula can be anchored in a hub or directly in the injection device without the use of a hub. If the needle cannula is anchored in a needle hub this needle hub can be either permanently or releasable coupled to the injection device.

As used herein, the term “drug” is meant to encompass any drug-containing flowable medicine capable of being passed through a delivery means such as a hollow needle cannula in a controlled manner, such as a liquid, solution, gel or fine suspension. Representative drugs includes pharmaceuticals such as peptides, proteins (e.g.

insulin, insulin analogues and C-peptide), and hormones, biologically derived or active agents, hormonal and gene based agents, nutritional formulas and other substances in both solid (dispensed) or liquid form.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

By the term “Pre-filled” injection device is meant an injection device in which the cartridge containing the liquid drug is permanently embedded in the injection device such that it cannot be removed without permanent destruction of the injection device. Once the pre-filled amount of liquid drug in the cartridge is used, the user normally discards the entire injection device. This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

The term “Permanently connected” or “permanently embedded” as used in this description is intended to mean that the parts, which in this application is embodied as a cartridge permanently embedded in the housing, requires the use of tools in order to be separated and should the parts be separated it would permanently damage at least one of the parts.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show a cross-sectional view of the needle assembly being connected to the injection device prior to breaking the sterility barrier.

FIG. 2 show a perspective view of the needle assembly being connected to the injection device prior to breaking the sterility barrier.

FIG. 3 show a cross sectional view of the needle assembly with the sterility barrier broken.

FIG. 4 show a perspective view with the sterility barrier disclosed separated from the needle hub.

FIG. 5 show a perspective view of an alternative sterility barrier.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

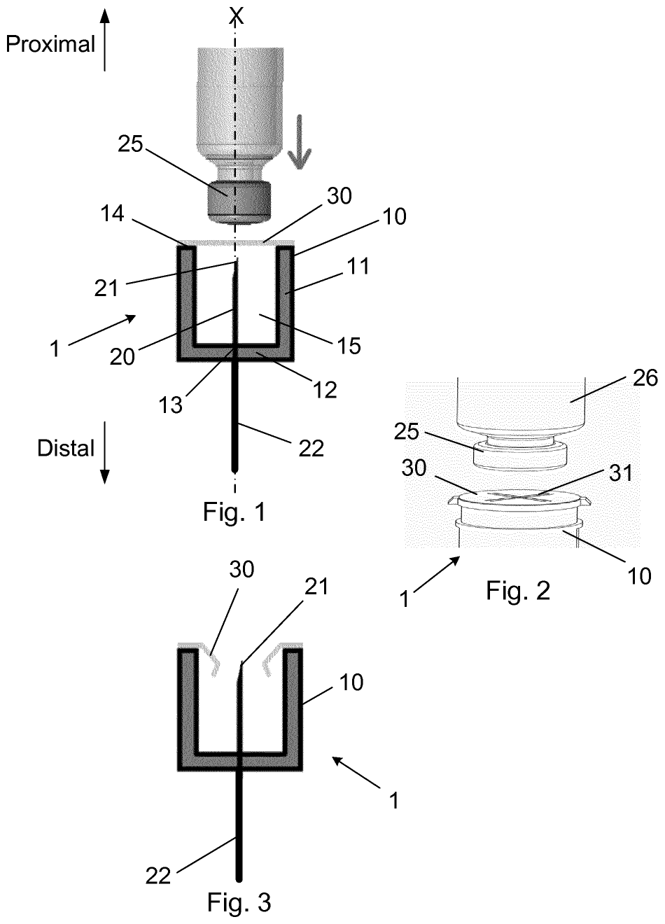

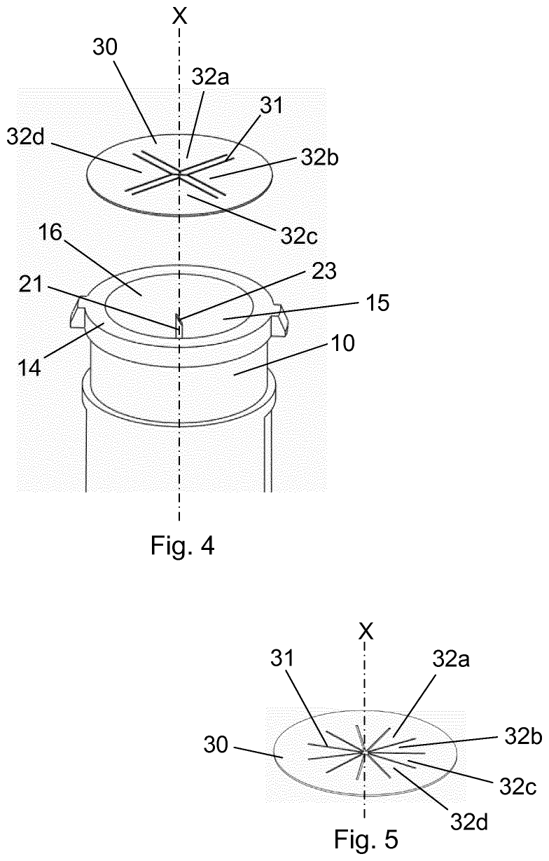

In that context it may be convenient to define that the term “distal end” in the appended figures is meant to refer to the end of the needle cannula doing the actual penetration of the skin of a user, whereas the term “proximal end” is meant to refer to the opposite end which enters into the cartridge. Distal and proximal is meant to be along an axial orientation extending along the central axis (X) of the injection device as also disclosed in FIG. 1 and in FIG. 4.

As disclosed in FIG. 1 the needle assembly 1 comprises a needle hub 10 having a surrounding wall 11 which in this example is disclosed as being cylindrical but which can take any form. This surrounding wall 11 is distally provided with a radial wall partition 12 which closes off the distal part of the needle hub 10.

The radial wall partition 12 further anchors a needle cannula 20 which in one example is glued into an anchor opening 13 in the radial wall partition 12. However, the needle cannula 20 could be attached to the needle hub 10 using alternative means of fastening.

The needle cannula 20 is usually made from a stainless steel and has a proximal end 21 pointing in the proximal direction from the anchor opening 13 in the radial wall partition 12 and a distal end 22 pointing in the distal direction.

The distal end 22 penetrates the skin of a user during an injection and the proximal end 21 penetrates into a cartridge 25 containing the liquid drug to be injected. The liquid drug is thus able to flow from the cartridge 25 and into the user through the lumen 23 of the needle cannula 20.

As seen in FIG. 2, the cartridge 25 is secured in an injection device 26 which can be any kind of delivery apparatus for delivering one or more doses of a liquid drug through the skin of a user.

The needle hub 10 forms a cup-shaped interior 15 which proximally have an opening 16 which is sealed by a sterility barrier 30. The sterility barrier 30 is typically made from paper or a suitable polymer and is connected to the proximal rim 14 of the needle hub 10 preferably by gluing. FIG. 4 discloses the needle hub 10 and the sterility barrier 30 before attaching the sterility barrier 30 to the rim 14 of the needle hub 10.

When the needle hub 10 is to be connected to an injection device the sterility barrier 30 is usually manually removed by the user. However, in some injection devices as e.g. known from WO 2014/064100 this is not possible since the needle assembly is provided in a concealed space.

In the example disclosed on FIG. 14 of WO 2014/064100, the proximal end of the needle cannula is protected by a sterility barrier made by latex and sealed to the needle hub. However, when the proximal end of this needle cannula is penetrated into the cartridge, the proximal end of the needle cannula is forced directly through the sterility barrier with the result that fragments of the sterility barrier can be caught inside the lumen of the needle cannula.

In order to avoid such fragments to enter into the lumen 23 of the needle cannula 20 upon penetration of the sterility barrier 30, the sterility barrier 30 is preferably provided with a plurality of weakened tear lines 31.

These tear lines 31 do not completely penetrate the thickness of the sterility barrier 30 to thereby maintain the sterility of the interior 15 of the needle hub 10. However, the tear lines 31 have a depth sufficient for the sterility barrier 30 to break in a predetermined pattern.

As seen in FIG. 2 these tear lines 31 can form a cross such that the breakable area is divided into four flaps 32a,b,c,d each having a triangular shape. The centre of the cross lies in the central axis X of the injection device (and of the needle assembly). Henceforth when the needle assembly 1 is connected to the injection device 26, the breakage of the sterility barrier 30 follows the cross of tear lines 31 and each of the four flaps 32a,b,c,d folds inwardly into the interior 15 of the needle hub 10 without contacting the proximal end 21 of the needle hub 10. It is thus prevented that fragments of the sterility barrier 30 is cut out and entered into the lumen 23 of the needle cannula 20.

The tear lines 31 can also as better seen in FIG. 4 be made as parallel lines which together forms a cross. Another alternative is depicted in FIG. 5. Herein the tear liens 31 are formed as multiple lines in a star-shaped pattern around the centre axis X such that each of the flaps 32a,b,c,d etc. are formed as triangular flaps 32a,b,c,d etc. with the tip of each flap 32a,b,c,d etc. laying in the centre line X.

The tear lines 31 can also have different depth such that one specific tear line 31 e.g. breaks before another tear line 31. In that way it is possible to predetermined the pattern in which the matrix of tear lines 31 breaks.

Some preferred embodiments have been shown in the foregoing, but it should be stressed that the invention is not limited to these, but may be embodied in other ways within the subject matter defined in the following claims.

1. A needle assembly, comprising
a needle hub having a surrounding wall with a distal part and a proximal part,
the distal part of the surrounding wall having a radially closed wall partition securing a needle cannula such that a distal end of the needle cannula extend distally from the radially closed wall partition and a proximal end extend proximally from the radially closed wall partition,
the proximal part of the surrounding wall having an opening sealed by a sterility barrier and wherein,
the surrounding wall, the radially closed wall partition and the sterility barrier together defined an interior being sterile and housing the proximal end of the needle cannula,
and wherein the sterility barrier is provided with a matrix of tear lines to enhance puncture of the sterility barrier and wherein the matrix of tear lines consist of a plurality of individual tear lines defining a plurality of flaps which flaps are bendable into the interior of the needle hub upon puncture of the sterility barrier.

2. The needle assembly according to claim 1, wherein the flaps has a triangular shape with one angle of the triangle being position in the center (X) of the needle hub.

3. The needle assembly according to claim 1, wherein three, four or more flaps are formed.

4. The needle assembly according to claim 1, wherein each of the tear lines in the matrix has a length sufficient for each flap to remain out of contact with the proximal end of the needle cannula when the flaps are bended into the interior of the needle hub.

5. The needle assembly according to claim 1, wherein the tear lines are carved or embossed into the sterility barrier.

6. The needle assembly according to claim 1, wherein the sterility barrier is made from paper, a metallic foil or from a polymeric foil.

7. The needle assembly according to claim 1, wherein the sterility barrier is connected to the proximal part of the needle hub.

8. The needle assembly according to claim 1, wherein the sterility barrier defines a thickness.

9. The needle assembly according to claim 8, wherein the thickness of the sterility barrier comprises of one or more layers.

10. The needle assembly according to claim 8, wherein the tear lines do not extend through the thickness of the sterility barrier.

11. The needle assembly according to claim 1 wherein, the depth of the tear lines differentiates.

12. The needle assembly according to claim 1 mounted onto an injection device comprising a cartridge by penetrating the injection device and/or the cartridge through the sterility barrier of the needle assembly such that the proximal end of the needle cannula connects to the cartridge without the proximal end of the needle cannula contacting the sterility barrier.
